(19) United States
(12) Patent Application Publication
(10) Pub. No.: US 2023/0235329 A1
(43) Pub. Date: Jul. 27, 2023

(54) ANTISENSE OLIGONUCLEOTIDES (ASO) FOR EFFICIENT AND PRECISE RNA EDITING WITH ENDOGENOUS ADENOSINE DEAMINASE ACTING ON RNA (ADAR)

(71) Applicant: Eberhard-Karls-Universität-Tübingen, Tübingen (DE)

(72) Inventors: Thorsten Stafforst, Tübingen (DE); Ngadhnjim Latifi, Tübingen (DE); Laura Sophia Pfeiffer, Ostfildern (DE)

(21) Appl. No.: 18/071,999

(22) Filed: Nov. 30, 2022

(30) Foreign Application Priority Data
Nov. 30, 2021 (EP) EP21211372.4

(51) Int. Cl. C12N 15/113

(52) U.S. Cl. CPC C12N 15/113; C12N 2310/11; C12N 2310/321; C12N 2310/315

(57) ABSTRACT

The present invention relates to a chemically modified oligonucleotide for use in site-directed A-to-I editing of a target RNA inside a cell with endogenous ADAR, comprising a sequence with a length of 11 to 100 nucleotides capable of binding to a target sequence in the target RNA, with a Central Base Triplet of 3 nucleotides with the central nucleotide opposite to the target adenosine in the target RNA, which is to be edited to an inosine, whereby the core sequence has the following Formula I:

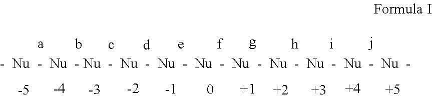

wherein Nu stands for a nucleotide having a sugar moiety which may be modified, the numbers below the nucleotide sequence designate the position of the nucleotides adjacent to the central nucleotide of the Central Base Triplet having the number 0 whereby the negative numbers designate the 5′ end and the positive number designate the 3′ end of the oligonucleotide and wherein a-j designate the nature of the linkage between the single nucleotides whereby at least linkages a, d, and e are phosphorothioate linkages and whereby at least 2 linkages are a phosphate linkage(s).

### REFERENCE TO RELATED APPLICATION

This application claims priority to European Patent application No. EP21211372.4, filed Nov. 30, 2021, the entire content of which is incorporated herein by reference.

### SEQUENCE LISTING

The instant application contains a Sequence Listing which has been submitted electronically as a file in XML format and is hereby incorporated by reference in its entirety. Said XML format file, created on Feb. 10, 2023, is named 2023-2-10 Sequence_Listing_Amended.XML and is 60,164 bytes in size.

### BRIEF DESCRIPTION OF THE DRAWINGS

FIGS. 1A and 1B show the effects of the phosphorothioate optimization on stability and on editing efficacy wherein the central core has been modified. In FIG. 1A the central core sequence and the phosphorothioate modifications are shown. Moreover, FIG. 1A shows the stability of the construct and the editing efficacy of each construct. It can be clearly seen that by increasing the number of the phosphorothioate linkages the stability can be substantially increased, whereby, however, the editing efficacy is reduced (FIG. 1A).

In FIG. 1A an oligonucleotide having phosphorothioate linkages at positions 1 (a) and 10 (j) only was used [v117.26]. Although the editing efficacy was rather high (59.2%±14) the stability (t₅₀ (100% FBS)) was only 30 h.

FIG. 1A shows also an oligonucleotide having phosphorothioate linkages at positions 1 (a), 7 (g), 8 (h), 9 (i) and 10 (j) [v117.27]. Although the stability against degradation (t₅₀) was improved to 40 h, the editing efficacy was reduced to 33.0%.

FIG. 1A shows also a construct having 6 phosphorothioate linkages at positions 1 (a), 2 (b), 3 (c), 4 (d), 5 (e) and 10 (j) [v117.28]. The editing efficacy improved to 50.3%, but the stability against degradation (t₅₀) was reduced to 20 h only.

FIG. 1A shows a further experiment [v117.29] wherein all linkages are phosphorothioate linkages. The editing efficacy was reduced to 32.0% only.

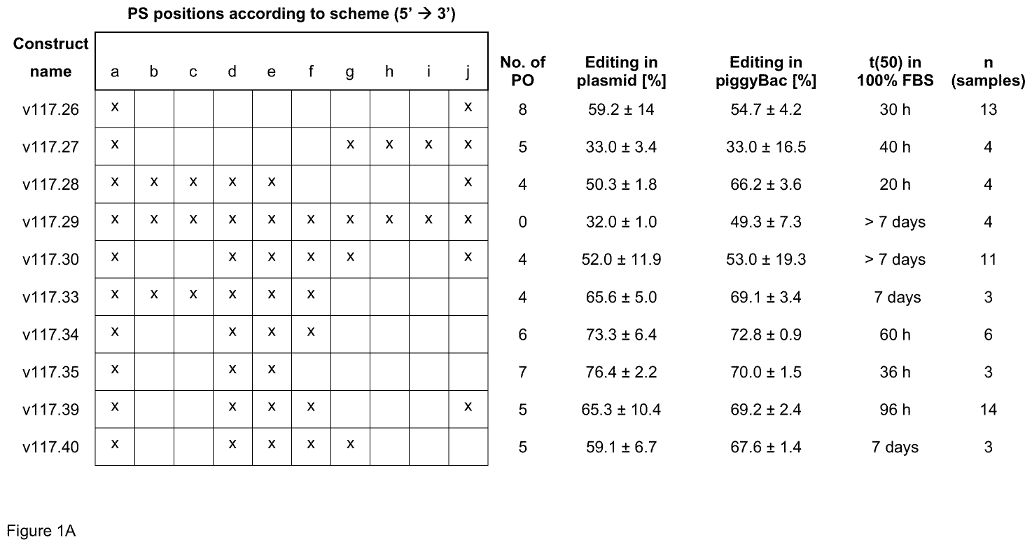

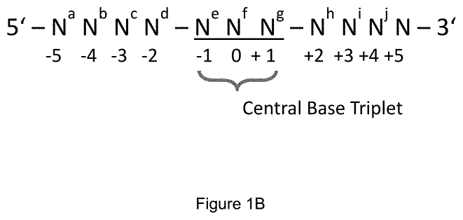

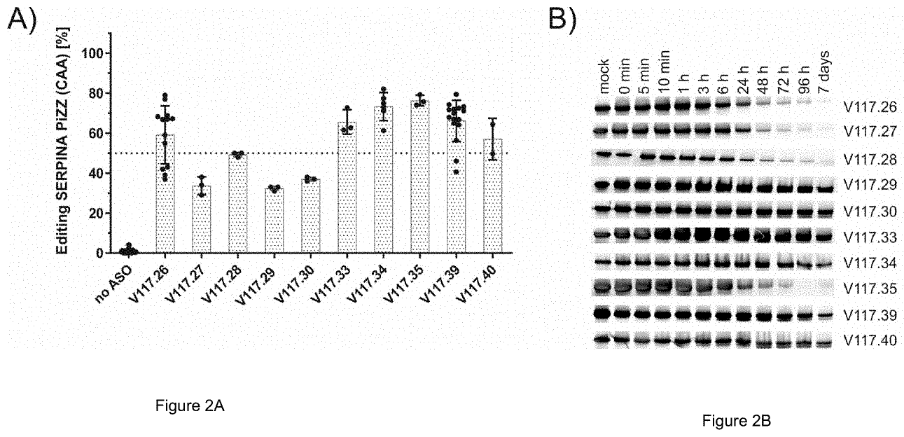

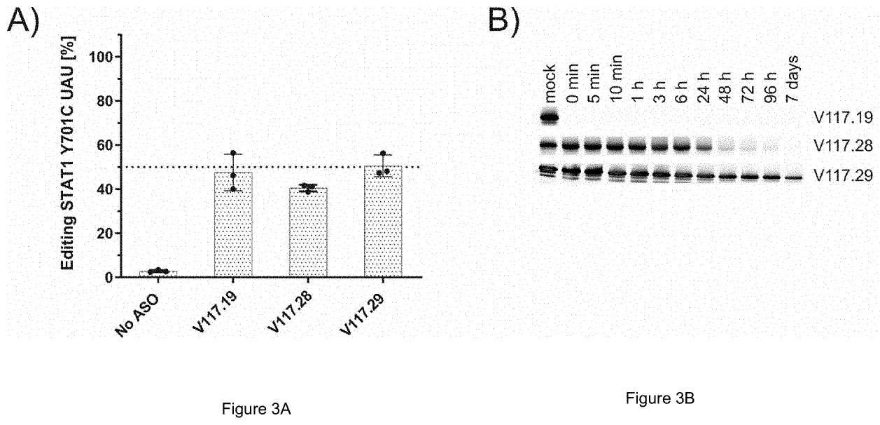

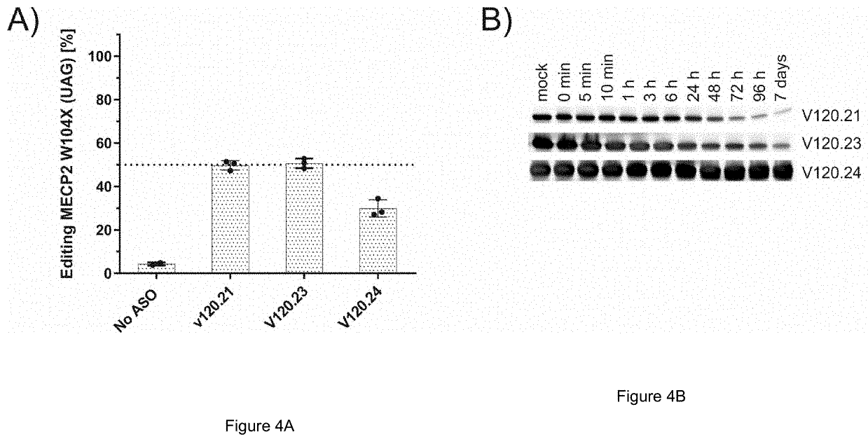

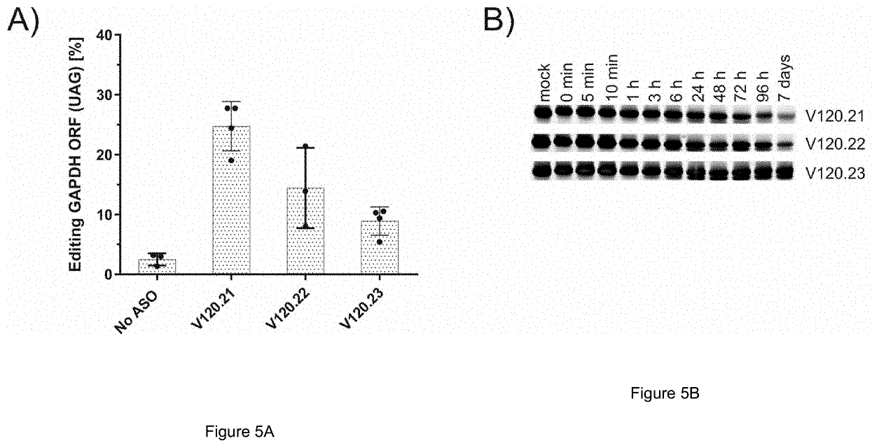

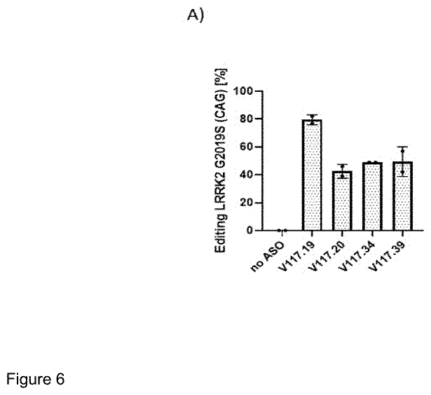

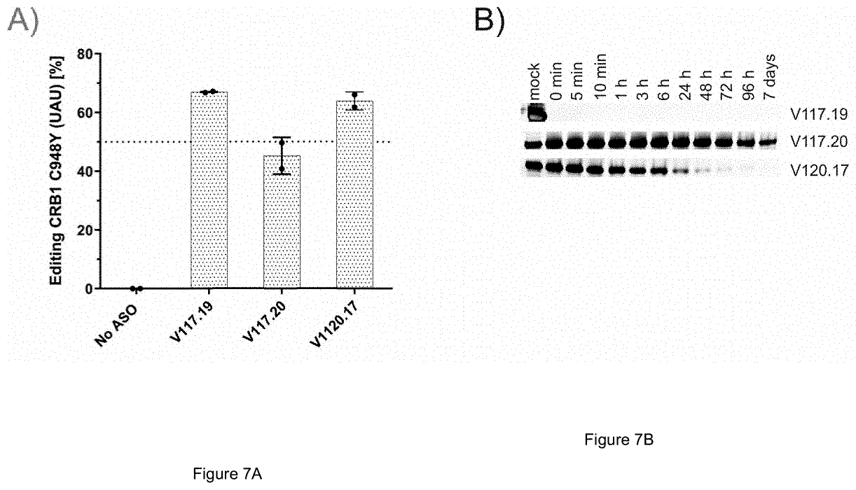

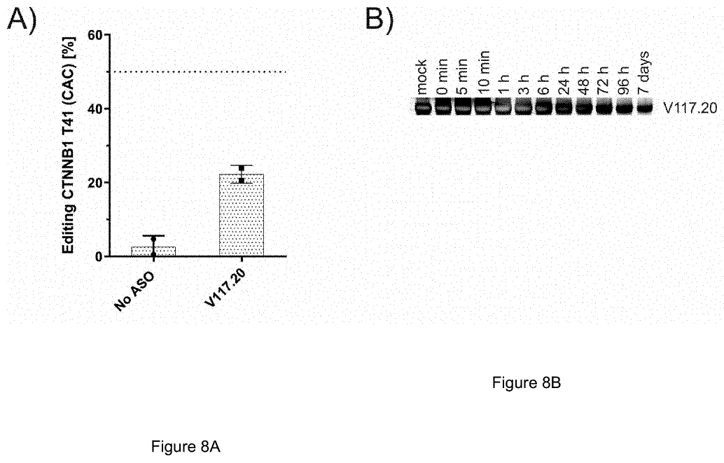

Contrary thereto, FIG. 1A shows an experiment [v117.30] wherein six out of 10 linkages are phosphorothioate linkages, namely at positions a, d, e, f, g and j. The editing efficacy improved to 52.0% and the stability (t₅₀) was >7 days.

The results shown in FIG. 1A whereby each linkage in the central core is a phosphorothioate linkage [v117.29] demonstrate that the stability of the construct is >7 days. Unfortunately, however, the editing efficacy is reduced to 32% only. This demonstrates that a pattern of the phosphorothioate linkage is to be observed when a reasonable editing efficacy shall be achieved.

On the other hand, the number of phosphorothioate linkages is not the only important factor since with only two phosphorothioate linkages (at positions 1 and 10) a stability with a t₅₀ (100% FBS) of 30 h could be achieved [v117.26].

In FIG. 1A and FIG. 1B the positions of the phosphorothioate linkages in the relevant samples are shown together with the editing efficacy and the stability. FIG. 1A shows that the best balance between high editing efficacy and high stability against degradation was obtained with sample designated v117.39. In this sample the phosphorothioate linkages in the core structure are located at a (1), d (4), e (5), f (6) and j (10). This pattern of the phosphorothioate linkages is especially preferred according to the present invention.

FIG. 1A shows the precise positions of the phosphorothioate linkages in constructs targeting the SERPINA1 E342K mutation as further explained in Example 1. The editing efficacies are shown from two different model systems (plasmid and piggyBac). The half-life of the constructs was measured in 100% FBS (t(50)). n designates the number of samples.

FIG. 1B shows the orientation of the bonds between the nucleotides with designations a-j. The central base triplet is highlighted.

FIGS. 2A and 2B show the editing yield results of the experiments performed in Example 1. The editing results are shown in FIG. 2A whereas the serum half-lives of the constructs in Example 1 are shown in FIG. 2B.

FIG. 3A shows the editing efficacy results of the experiments performed in Example 2 while the corresponding serum half-lives in 100% FBS are shown in FIG. 3B.

FIG. 4A shows the editing efficacies of the constructs targeting the disease-causing W104X mutation in murine MECP2. FIG. 4B shows the serum half-lives.

FIGS. 5A and 5B show the results of Example 4. The editing efficacies of the constructs are provided in FIG. 5A whereas the stabilities shown in FIG. 5B.

FIG. 6 shows the editing results of Example 5.

FIGS. 7A and 7B show the results of Example 6. FIG. 7A shows the editing efficacy and FIG. 7B shows the stability.

FIGS. 8A and 8B show the results of Example 7 whereby the optimized phosphorothioate design according to Example 1 (V117.39) was transferred to an oligonucleotide targeting the T41 site in murine CTNNB1. The Figure shows that the disclosed pattern can be applied also to other targets.

### DETAILED DESCRIPTION OF THE INVENTION

The present invention concerns processes and chemically modified nucleic acids for use of site-directed editing of a target RNA. In view of the tremendous progress made by molecular biology, it is possible to alter the genetic information of cells. While the editing of DNA usually leads to stable modification of the genetic information of the cells, it is sometimes interesting not to change the DNA but to change the genetic information in the (m)RNA. A major advantage of editing (m)RNA over DNA is on the one hand the dose-dependency of the editing yield and on the other hand the reversibility of the treatment. By regulating the concentration of the chemically modified nucleic acid in the cells where the target RNA shall be edited, a dependency on the editing yield and thus the amount of modified protein after translation of the target RNA can be achieved. What is more, the treatment is reversible since the editing of the target RNA is halted when the chemically modified nucleic acid is no longer present in the relevant cell, and the edited RNA is replaced by newly transcribed unedited RNA.

Editing of RNA molecules according to the invention is mediated by enzymes belonging to the family of adenosine deaminases acting on RNA (ADARs). ADARs are members of an enzyme family that catalyze the deamination of adenosine (A) to inosine (I) in double-stranded RNA (A-to-I RNA editing). In the course of this enzymatically catalyzed reaction adenosine is changed via a hydrated intermediate to inosine. While guanosine can form three hydrogen bonds to the complementary base cytidine, inosine can form only two hydrogen bonds to cytidine. The translational machinery reads inosine as a guanosine. Therefore, ADARs have the effect of introducing a functional adenosine to guanosine mutation on the RNA level.

A requirement for a deaminase belonging to the ADAR family to act on RNA, in particular mRNA, is that a double strand is formed. Therefore, it is required to provide a complementary nucleic acid (in the following: oligonucleotide or oligoribonucleotide) which can form a double-stranded molecule on which the ADAR can act.

The present invention discloses chemically modified nucleic acids which can cause a functional change from an adenosine (A) to a guanosine (G). Depending on the sequence of the RNA such a change can have dramatic effects. It may either cure point mutations which have deleterious effects of the protein encoded by the mRNA or other amino acids may be incorporated in the translated protein by substitution. Strong effects can be observed, however, when stop codons (UAA, UAG, UGA) are edited or splice sites.

A substantial advantage of the chemically modified oligonucleotides of the present invention is that such off-target edits are reversible and the danger of devastating side effects is less likely. Moreover, therapy can be stopped and reverted if necessary. Due to the better safety profile, the temporary and limited manipulation of human genetic information at the RNA level may become broadly applicable and may be expanded to medical indications whereby genome editing on the DNA level may be dangerous due to unforeseeable and irreversible side effects.

There are several ADAR enzymes expressed across human tissues which enable the conversion of adenosine to inosine, which in turn is biochemically read in translation as guanosine. Several ADARs are known in the art. ADAR has been found in Xenopus levis but also in human and murine cells. While all three human ADARs share a common C-terminal deaminase domain, only ADAR 1 and ADAR 2 revealed to be catalytically active. ADARs share a common functional domain which is the double-stranded RNA binding domain (dsRBD). While ADAR 1 contains three, ADAR 2 and ADAR 3 share only two dsRBDs. For ADAR 1 two isoforms are known. The short constitutively expressed 110 kDa ADAR 1 is the p110 isoform whereas the longer 150 kDa ADAR 1 is the p150 isoform, which is expressed from an alternative interferon inducible promoter. According to the present knowledge, ADAR 2 predominantly edits coding sites in the brain. The ADAR 1 is the major enzyme for editing non-coding sites.

For an efficient editing of RNA it is necessary that the ADAR is directed to specific target sites on the mRNA transcript. Previous attempts in the prior art utilized a specific, loop-hairpin structured ADAR recruiting moiety derived from natural, cis-acting ADAR recruiting sequences to direct the deaminase activity of ADARs to specific sites, thereby bringing the deaminase activity of ADAR to the correct position on the mRNA molecule to be edited. Such artificial nucleic acids for site-directed RNA editing are disclosed in WO 2020/001793. The artificial nucleic acid disclosed in the prior art comprises a targeting sequence, which in turn comprises a nucleic acid sequence complementary or at least partially complementary to a target sequence in the target RNA and a recruiting moiety for recruiting a deaminase.

The chemically modified nucleic acids according to the present invention differ from the nucleic acid oligonucleotides disclosed therein insofar that they do preferably not have a loop-hairpin structured recruiting moiety specifically for recruiting a deaminase. The chemically modified nucleic acids of the present invention use another strategy than the constructs known from the prior art. It is well-known that RNAs are highly unstable due to the ubiquitous presence of different RNA digesting enzymes, in particular RNase A and H.

Editing of RNA with the constructs of the prior art is only achieved by recruiting the deaminase with the help of the recruiting moiety such as an imperfect hairpin for endogenous ADAR, or other oligonucleotide motifs, such as a BoxB or MS2 motif. When the chemical modifications according to the present invention are used, a separate recruiting moiety motif may no longer be necessary. However, in some embodiments such motifs may be present in order to improve the efficacy. Generally the recruiting moiety guides the deaminase to the desired site of action, namely the target adenosine which shall be converted to an inosine, but functionally a guanosine.

The chemically modified nucleic acids according to the present invention do not necessarily have a loop-hairpin structured recruiting moiety for a deaminase. Instead, the chemically modified nucleic acids of the present invention form an RNA duplex to which the ADAR enzyme adheres, whereby the editing efficiency is increased. The latter is achieved by using chemically modified nucleic acids of a specific optimal chemical modification pattern over its whole length. It is an important aspect of the present invention that the chemical modification of the ASO is not limited to the central triplet but it extends over the flanks adjacent to the central triplet.

The three central bases of the target RNA sequence comprises an adenosine flanked by one nucleotide on both sides, and will be further referred to as the Central Base Triplet. The sequence complementary to the Central Base Triplet in the chemically modified oligonucleotide of the present invention is important with regard to its specific chemical modification. In order to enable a functional change on the translational level of the mRNA (editing), it is required that the oligonucleotide according to the invention allows the editing of the mRNA. It is essential that the oligonucleotides according to the invention are on the one hand sufficiently stabilized against degradation (caused e.g. by RNase) which can be achieved by chemical modification of the oligonucleotide, in particular by modification of the sugar moieties of the oligonucleotide and in particular through modifications of the phosphate backbone preferably by replacing phosphate linkages by phosphorothioate linkages.

On the other hand, the chemical modification must allow the editing of the RNA molecule. If the chemical modification of the oligonucleotide is too extensive, the efficacy of editing is reduced to an unacceptable level. Therefore, the modification of the oligonucleotide must follow the guidelines as described herein in order to obtain an optimal editing efficacy.

EP 3 507 366 discloses chemically modified single-stranded RNA-editing oligonucleotides for the deamination of a target adenosine by an ADAR enzyme whereby the central base triplet of 3 sequential nucleotides comprises a sugar modification and/or a base modification. The flanking regions, in all embodiments (FIG. 2 of said prior art), are uniformly modified with blocks of 2″ methylation at the ribose units plus a small number of additional terminal phosphorothioate linkages.

However, it was found that such uniform and block-wise 2″-O-methyl modification of the nucleic acids as used in the prior art above leads to a strong loss of editing activity with natural ADAR enzymes at their endogenous expression levels. This is in accordance with a negative effect of bulky 2″-modifications on the binding of double-stranded RNA binding domains to dsRNA substrates. In general 2″-F and mixtures of 2″-F and 2″-OMe are particularly well accepted and still have a good stabilizing effect against nuclease digestion when they are placed at all pyrimidine bases on the nucleic acids. However, in the Central Base Triplet 2″-F and in particular 2″-O-methylation had a strongly negative effect on editing yield, which is in accordance to literature. However, it was also found that deoxyribose at all three positions of the Central Base Triplet is well tolerated and provides substantial stabilization against nuclease digestion. In a preferred embodiment of the present invention the three sugar units of the oligonucleotide complementary to the central triplet are desoxyribose units.

The chemically modified nucleic acids are suitable for use in site-directed editing of a target mRNA. The chemically modified nucleic acids comprise a sequence, which is completely complementary to a target sequence in the target mRNA with the exception of the central nucleotide of the Central Base Triplet, which is opposite to the target adenosine. The central nucleotide of the Central Base Triplet is typically a cytosine or a derivative thereof, but can also be a nucleobase analogue, typically built on an N-heterocyclic compound, and replaces the normally complementary thymidine or uracil and usually improves editing site recognition by ADAR. By action of the adenosine deaminase, the target adenosine is functionally changed to a guanosine post-transcriptionally. Therefore, the sequence of the nucleotides is always complementary to the target region of the mRNA with the one exception as previously described to improve the recognition of the targeted adenosine by ADAR within the dsRNA formed when the administered oligonucleotide hybridizes with the target RNA. Important is furthermore the pattern of the modification of the oligonucleotide, in particular of the sugar moieties and the linkages there between.

The principle of the present invention is based on the fact that the chemically modified nucleic acids must be stable for a sufficient period of time in order to allow the editing of the mRNA. Normally, RNA molecules are degraded very quickly in the cells. Therefore, the nucleic acids are chemically modified, whereby the modification must occur to such an extent that the chemically modified nucleic acids survive for a sufficient time span in the cell, and the modifications simultaneously do not hinder recognition by ADAR. The modification of the oligonucleotides relates to the sugar moiety of the nucleotide. RNA bases are the unmodified moieties. The preferred modifications which stabilize the oligonucleotide are deoxy-ribose moieties or RNA bases with 2′-O-methyl or 2′-F modifications at the ribose moiety. Another important modification is the replacement of the phosphate bond between the sugar moieties by a phosphorothioate bond, whereby the percentage and the position of the phosphorothioate bonds in the core region plays a decisive role.

There are many chemical modifications of the oligonucleotides known which have an influence on the properties of the oligonucleotides. The modifications of the sugar residue are mainly substitutions at the 2′-position whereby 2′-F, 2′-OMe and 2′-NH₂ are known as well as conformationally locked sugars like LNA, cEt and/or ENA. Such modifications increase the nuclease resistance and maintain the compatibility of the ASO with many biochemical activities. A modification which is particularly relevant for the present invention is the phosphodiester linkage whereby the phosphate residue is modified to a phosphorothioate, wherein an oxygen atom of the phosphate group is replaced by a sulphur atom. The stereochemistry may have an influence on the oligonucleotide's property. Such modification increases the resistance against degradation by nucleases but maintain the compatibility of the ASO with many biochemical activities.

In this regard, not only the resistance against degradation by nucleases is relevant, but the editing efficacy is of utmost importance. Therefore, a balance between sufficient resistance against degradation by nucleases coupled with a sufficiently high editing efficacy is desired. The oligonucleotides according to the present invention have specific patterns of the phosphorothioate linkages which provide such advantageous properties.

In the course of the invention, it has been found that the artificial nucleic acid (oligonucleotide) has a length of 15 to 80 nucleotides, preferably 25 to 65 nucleotides, more preferably 30-60 nucleotides. Nucleic acids of such length are designated as oligonucleotides in the present application.

The chemically modified nucleic acids (oligonucleotides) according to the invention have a sequence which is complementary to the corresponding sequence in the target mRNA with a complementarity of nearly 100%. In some embodiments the complementarity of the chemically modified oligonucleotides to the corresponding sequence in the target mRNA is at least 85%, preferably 95% complementarity. While full complementarity is optimal for the hybridization process, natural ADAR substrates often contain a small number of mismatches and/or bulges, which assist the editing by allowing structural perturbations of the double-stranded substrate to improve substrate recognition by the double-strand RNA binding domain or inside the active site of the deaminase.

The chemically modified oligoribonucleotide according to the present invention comprises a core sequence of formula I:

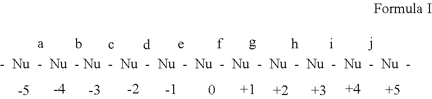

In this formula I there is a Central Base Triplet of three nucleotides, whereby the central nucleotide is designated by “0”. The nucleotide designated as “0” and the two nucleotides directly adjacent to nucleotide “0” having the number −1 and +1 are designated as a Central Base Triplet, whereby the central nucleotide designated as “0” is directly opposite to the target adenosine in the target RNA. The nucleotide of formula I is flanked at the 5′- and (adjacent to nucleotide −5) and at the 3′-end (adjacent to nucleotide +5) with further oligonucleotide sequences, which may have either the same length or different lengths.

In the Central Base Triplet of the chemically modified nucleic acids according to an embodiment of the invention there is a nucleoside carrying an N-heterocyclic base, a pyridine or pyrimidine derivative, more preferably a cytosine nucleoside or a derivative thereof, which is opposite of the target adenosine in the target (m)RNA. In a particularly preferred embodiment this nucleoside and the 5′ and 3′-singular neighbouring nucleotides comprise at least one modified nucleoside, more preferred two modified nucleosides, even more preferred three modified nucleosides, having a substituent at the 2′ carbon atom whereby the substituent is either 2′-fluoro or 2′-O-methyl. In the most preferred embodiment all three bases are 2′-desoxyribose moieties.

When a 5″-CAN codon (targeted A underlined, N=any nucleobase) is targeted in a target (m)RNA, then the Central Base Triplet of the chemically modified nucleic acid according to the invention contains a 2′-deoxy-inosine or any nucleotide harbouring a hypoxanthine nucleobase or a derivative thereof, which pairs with the cytosine base 5′-adjacent to the targeted adenosine. Preferably, the 2′-deoxy-inosine is placed in a Central Base Triplet containing two, more preferably three 2′-deoxynucleotides.

Since the chemically modified nucleic acids according to the present invention show increased stability against degradation and an optimal chemical modification pattern to bind ADARs, the nucleic acids according to the present invention preferably do not necessarily have a specific loop-hairpin-structured recruiting moiety which attracts the deaminase.

In one embodiment of the present invention the chemically modified nucleic acids are symmetrical, which means that the two nucleotide sequences adjacent to the Central Base Triplet have the same length. When the oligonucleotide has for example 59 nucleotides there are 28 nucleotides on each side of the Central Base Triplet.

In another embodiment, the nucleic acids according to the present invention are not symmetrical which means that the two sequences flanking the Central Base Triplet have different lengths. The asymmetric design enables a more flexible use of the sequence space around the target. Furthermore, it was found that the asymmetric design can enhance editing yields in short sequences of the nucleic acid, e.g. 45 nt, compared to the symmetric design, provided that the nucleic acid is shortened at the correct terminus. Preferably, the flanking sequence 5′ to the Central Base Triplet is longer than the flanking sequence 3′ in asymmetric embodiments. Preferred embodiments comprise at least 4 nt, more preferred at least 9 nt at the 3′ flanking sequence, and comprise at least 19 nt, more preferably at least 28 nt, most preferably at least 33 nt at the 5′ flanking sequence.

The nucleic acids according to the present invention comprising the core sequence according to formula I are linked via phosphorothioate linkages to a percentage of at least 40%, more preferably more than 50% and especially preferred 60%. The phosphorothioate pattern in the core sequence of formula I is of utmost importance. The linkages a, d and e are always phosphorothioate linkages whereby in addition thereto up to three linkages selected from the group consisting of linkages b, c, f, g and j may also be phosphorothioate linkages. It is, however, excluded that all linkages a-j are phosphorothioate linkages. In especially preferred embodiments the linkage f is a phosphorothioate linkage.

In preferred embodiments of the present invention the sequences flanking the core sequence of formula I comprise at least 10, more preferably at least 15, most preferably 20 or more nucleoside linkages which are phosphorothioate linkages with little discontinuity, more preferably without any discontinuity, starting from a terminus (5′ or 3′) of the nucleic acid. In another embodiment of the invention said blocks of preferably continuous phosphorothioate linkages are placed on both flanks of the nucleic acid starting from both termini (5′ and 3′).

In the core region according to formula I of the oligonucleotide there are, however, less than 60%, particularly preferred less than 50%, preferably less than 40% of the linkages phosphate linkages whereby a specific pattern has to be observed. The linkages h and i are always phosphate linkages. In preferred embodiments of the present invention, not only linkages h and i are phosphate linkages, but also linkages b and/or c may be phosphate linkages.

In especially preferred embodiments, linkages a, d and e are phosphorothioate linkages whereas linkages h and i are phosphate linkages. In preferred embodiments the core sequence of formula I comprises preferably up to six out of ten phosphorothioate linkages.

The chemically modified nucleic acids according to the present invention are substantially more stable against degradation usually effected by RNases which in turn allows them to be longer present in the cells wherein the (m)RNA should be edited. Without wishing to be bound to a theory, it is assumed that—since the lifetime of the chemically modified nucleic acids is increased in the environment of the cells—no recruiting moiety for recruiting the deaminase is required, because the ADARs can act on the mRNA due to the longer stability of the double strand.

Biological reactions are frequently time-dependent. There is a large variety of different RNA molecules in cells of vertebrates which are subject to a permanent and quick turnover. RNA molecules are frequently degraded by different RNases. Therefore, the use of RNA molecules for therapeutic purposes is frequently limited by the rapid degradation of the RNA molecules. Since the situation in vivo is usually different from the situation in vitro, where test systems with cell cultures are used, the stability of the molecules used for therapeutic purposes may be decisive for the success of the treatment.

The chemically modified nucleic acid molecules according to the invention provide a good balance of editing capability and sufficient stability in the cells whereby even the condition in the endosome can be tolerated. The chemically modified oligonucleotides according to the present invention are furthermore capable of gymnotic uptake and show an editing efficiency which is acceptable.

Best results with the chemically modified nucleic acids according to the invention can be achieved when preferably several, at least two and more preferred at least three of the following features are realized in the oligonucleotide, namely:
in the central core sequence of formula I up to 4 to 6 of the linkages a-j are phosphate linkages whereas the remainder are phosphorothioate linkages;
at least one DNA sugar nucleoside in the Central Base Triplet opposite to the adenosine to be deaminated;
stabilization of pyrimidine bases outside the Central Base Triplet by 2′-F or 2′-OMe modifications of the nucleoside ribose moieties in roughly equal stoichiometry, whereby a pattern is preferred that avoids blocks of 2″-OMe moieties;
stabilization of both termini by blocks of three nucleotides which have a double modification, namely a 2′-OMe modification on the sugar moieties and a phosphorothioate linkage.

The chemically modified nucleic acid molecules (oligonucleotides) of the present invention have the advantage that the molecule is sufficiently stable in a vertebrate organism so that the desired effect can be achieved. The molecules according to the present invention are stable against a degradation of different RNases for a sufficient period of time so that an effect can be seen.

Another advantage of the chemically modified nucleic acids of the present invention is that they can be brought directly to the target cells without specific vectors or other helping mechanisms like specific transfection methods. The chemically modified nucleic acids according to the present invention can act via gymnosis, meaning they can be applied directly to the target cells without helping means like vectors or other carriers.

A further advantage of the chemically modified nucleic acids according to the present invention is that they have a high efficiency of editing in clinically relevant targets. The modified nucleic acids can be introduced via gymnosis into the target cells and a comparatively high effect on the translational level in the target cells can be achieved.

Another advantage of the chemically modified nucleic acids according to the present invention is that an editing from A to I can be effected not only with comparatively easily editable targets like 5″UAG but also with more difficult triplets like 5″CAA.

The present invention relates to a chemically modified oligoribonucleotide for use in site-directed A-to-I editing of a target RNA inside a cell with endogenous ADAR, comprising a sequence with a length of 11 to 100 nucleotides, preferably 20 to 80 nucleotides, capable of binding to a target sequence in the target RNA, with a Central Base Triplet of 3 nucleotides with the central nucleotide opposite to the target adenosine in the target RNA which is to be edited to an inosine. The oligonucleotides have a core sequence having the following Formula I:

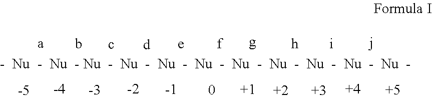

wherein Nu stands for a nucleotide having a sugar moiety which may be modified. The numbers below the nucleotide sequence designate the position of the nucleotides adjacent to the central nucleotide having the number 0 whereby the negative numbers designate the 5′ end and the positive number designate the 3′ end of the oligonucleotide. Nucleotide (0) and nucleotides (−1) and (+1) form the central base triplet. Letters a-j designate the linkage between the single nucleotides in the core sequence according to formula I. In the examples and the tables describing the used oligonucleotides a phosphorothioate linkage is designated by an “*”. Each nucleotide Nu may have independently from each other a meaning which differs with regard to base and sugar and modifications thereof.

The chemically modified oligonucleotides of the present invention have a total length ranging from 11 to 100 nucleotides whereby the length preferably ranges from 20 to 80 nucleotides. In a particularly preferred embodiment the chemically modified oligoribonucleotides according to the present invention range from 30 to 60 nucleotides which comprise the core sequence of formula I. The sequences flanking the core sequence having formula I may have the same length ranging from 9 nucleotides to 25 nucleotides. In alternative embodiments the strands flanking the core sequence may have different lengths.

In addition to the specific phosphorothioate pattern additional modifications may be used. Such modifications may be at the 2′-position of the sugar moiety. Purines and/or pyrimidines may be modified or not modified.

According to the present invention the core sequence has mandatory phosphorothioate linkages at positions a, d, and e. Furthermore, the present invention has mandatory regular phosphate linkages at positions h and i. In other words, five out of the ten linkages are defined to be either PS or regular phosphate. The remaining five linkages b, c, f, g, and j can be chosen from both PS and regular phosphate resulting in several preferred embodiments:

In one preferred embodiment the linkages at position f, g, j are phosphorothioate while linkages in position b, c are phosphate. The other five linkages a, d, e and h, i are as defined above. In another preferred embodiment the linkages at position b, c, f are phosphorothioate while linkages in position g, j are phosphate. The other five linkages a, d, e and h, i are as defined above. In another preferred embodiment the linkage at position f is a phosphorothioate while linkages in position b, c, g, j are phosphate. The other five linkages a, d, e and h, i are as defined above. In another preferred embodiment the linkages at position f, j are phosphorothioate while linkages in position b, c, g are phosphate. The other five linkages a, d, e and h, i are as defined above. In another preferred embodiment the linkages at position f, g are phosphorothioate while linkages in position b, c, j are phosphate. The other five linkages a, d, e and h, i are as defined above. In another preferred embodiment the linkages at position b, c, f, g, j are phosphate linkages. The other five linkages a, d, e and h, i are as defined above.

The chemically modified oligonucleotide of the invention may be formulated into a composition with any suitable excipient, in particular a pharmaceutically acceptable excipient.

The chemically modified oligonucleotide of the invention may be for therapeutic or diagnostic use, preferably for therapeutic use.

The chemically modified oligonucleotide of the invention may be for use in the treatment of a genetic disease or disorder. In particular, the genetic disease or disorder may be a metabolic disease, a cardiovascular disease, an autoimmune disease or neurological disease. In this context the present invention encompasses a method of treating such a disease or disorder by administering an effective amount of said chemically modified oligonucleotide to the subject in need thereof.

The present invention and preferred embodiments thereof are illustrated, but not limited to those depicted by the Examples and the Figures.

The Figures illustrate in particular preferred embodiments of the invention.

### Example 1: Optimization of PS-Positioning Near the Central Base Trip/Et on the Human SERPINA1 Gene at the Disease-Causing E342K Mutation Site

Long stretches of PS (phosphorothioate) linkages improve the stability and in turn, the bioavailability of the oligonucleotide. From a therapeutic perspective, this would mean that lower doses or less frequent treatment with a PS-linked construct would be sufficient for a desired effect compared to an analog phosphodiester (P0)-linked constructs. However, the simple exchange of all PO linkages by PS linkages proves detrimental to editing efficacy. Here, different placements of PS linkages within the 10 phosphodiester linkages around the Central Base Triplet are screened, an area that is particularly sensitive for PO/PS substitution in terms of editing efficiency and stability. The example is based on a therapeutically highly relevant substrate, the E342K mutation of the SERPINA1 gene, which is the underlying cause for the severe Z-phenotype of α-1-antitrypsin deficiencies, representing an unmet clinical challenge. A list of all oligonucleotide constructs used is provided in Table 1.

The editing yield results of Example 1 are shown in FIG. 2A), while the serum half-lives of the constructs in Example 1 are shown in FIG. 2B). First, it is shown that uniform placement of phosphorothioate (PS) linkages at all 10 positions strongly decreases editing efficiency (FIGS. 1A, 1B and 2A). Second, there are several positions, in particular a, d, and e, where PS linkages are very well accepted. Third, additional PS linkages can be added at specific positions, which can further improve stability and/or editing efficiency. Overall, optimal PS patterns are available that strongly improve the serum half-life of the oligonucleotides without losing significant editing yield (FIGS. 1A, 1B and 2A). The best solutions combine enhanced stability and enhanced editing yield.

2.5×10⁴ HeLa cells (Cat. No.: ATCC CCL-2) were seeded in a 24-well plate. After 24 h, cells were forward transfected with a plasmid containing the human SERPINA1 E342K mutated cDNA or the SERPINA1 healthy cDNA (“wildtype”). 300 ng plasmid and 0.9 μl FuGENE® 6 (Promega) were each diluted in 50 μl Opti-MEM and incubated for 5 min, then combined and incubated for an additional 20 min. The medium was changed, and the transfection mix evenly distributed into one well. 24 h after plasmid transfection, cells were forward transfected with 5 pmol construct/well and 1.5 μl/well Lipofectamine RNAiMAX Reagent (ThermoFisher Scientific). After 24 h, medium was changed. 48 h after transfection, cells were harvested for RNA isolation and sequencing.

As is shown in FIG. 2A, the placement of PS linkages at the observed linkage positions can have a strong influence on the editing levels. V117.26 (SEQ ID NO:1) contains PS linkages only at positions a and j. There are no stabilizing PS positions near the Central Base Triplet (CBT). Consequently, while the editing efficacy of the embodiment is >50%, the half-life in 100% FBS is only around 30 h.

When PS linkages are placed 3′ of the CBT, i.e. v117.27 (SEQ ID NO:2) and v117.29 (SEQ ID NO:4), editing levels drop by about 50% as compared to v117.26. Thus, placing PS linkages at positions h and i proves to impair editing strongly, while a PS linkage at position g only shows a small effect on editing, which is seen when comparing v117.39 (SEQ ID NO:9) to v117.40 (SEQ ID NO:10) and v117.30 (SEQ ID NO:5). Adding PS linkages to all positions, as is seen for v117.29 (SEQ ID NO:4), strongly increases the half-life of the ASO in 100% FBS (>7 days), but at the cost of strongly decreased editing yields compared to v117.26. Thus, a precise placement of PS linkages which increases serum half-life but does not impair editing efficacies is desirable. This would include avoiding the placement of PS linkages at positions h and i.

In contrast thereto, when PS linkages are placed at the linkages 5′-adjacent to or inside of the CBT, editing yields stay similar to those observed for v117.26, e.g. as seen in version v117.28 (SEQ ID NO:3), v117.30 (SEQ ID NO:5) and v117.40 (SEQ ID NO:10). For some embodiments, editing yields were even improved, as seen for v117.33 (SEQ ID NO:6), v117.34 (Seq. ID No. 7), v117.35 (SEQ ID NO:8), and v117.39 (SEQ ID NO:9). These embodiments also show that while PS linkages at positions b and c do not impair editing, they also do not seem to be as strongly necessary for the stability of the ASO. Thus, their role for the overall construct performance is somewhat neutral.

However, especially concerning the serum half-life of the ASO, the PS linkages at the CBT (positions d-g) are essential, as seen e.g. in the embodiment v117.28 (SEQ ID NO:3) compared to v117.30 (SEQ ID NO:5). Both versions have the same amount of linkages (six PS, four PO), but the linkages at the CBT make the ASO significantly more stable (>7 days vs. only 20 h in 100% FBS) than the linkages 5′-adjacent to the CBT. This stresses the importance of the precise positioning of the PS linkages within the ASO, which can be underlined by comparing the serum half-life and editing efficacies of ASOs with the same overall number of PS linkages, but with a different arrangement. For example, the embodiments v117.28 (Seq. ID No. 3), v117.30 (SEQ ID NO:5) and v117.33 (SEQ ID NO:6) all have six PS linkages. However, editing efficacies of v117.28 and v117.30 are similar (around 50%), while v117.33 has a higher editing efficacy (ca. 66%). Furthermore, the serum half-lives of v117.30 and v117.33 are significantly higher (>7 days) compared to v117.28 (ca. 20 h). Overall, this would make v117.33 the embodiment with the most favorable positioning with six PS linkages in terms of the combination of high editing efficacy and a high serum tolerability. Similarly, the embodiments v117.27 (SEQ ID NO:2), v117.39 (SEQ ID NO:9) and v117.40 (Seq. ID No. 10) can be compared for the most favorable positioning of five PS linkages, with the latter two clearly outcompeting v117.27 (SEQ ID NO:2). An overview of the different embodiments alongside their precise PS-linkage placements, corresponding editing yields and 100% FBS half-lives (t50) is provided in FIGS. 1A and 1B.

Consequently, this makes the PS linkages at positions a, d and e the most essential for a prolonged half-life in 100% FBS without impairment of the editing yields of the construct. However, introducing PS linkages at positions b, c, f, and j can further improve these qualities of the construct. A PS linkage at position g can also improve the serum half-life of the construct, but will likely slightly affect the editing efficacy. However, PS linkages should not be placed at positions h and i, which are clearly detrimental to the editing efficacy of the construct. The positions of the PS linkages from the embodiment v117.39 (SEQ ID NO:9) were chosen as the most preferred balance between high editing yields and a long half-life in 100% FBS and further tested in other targets (see further Examples below). The corresponding positions are a, d, e, f and j.

TABLE 1
Construct sequences and modifications used in Example 1.

| | | SEQ | |
|---|---|---|---|
| Construct | | ID | FIG. |
| Name | Construct Sequence (5′ to 3′ direction) | NO: | No. |
| V117.26 | mC*mA*mU*G*G* fC*mC* fC* mC*A*G* fC* A*G* mC* fU* | 1 | 1A, |
| (disclosed | mU* fC* A*G* mU* fC* mC* fC* mU* fU mU fC dTdCdl mU | | 1B |
| in EP | fC G*A* mU* G*G* fU* mC* A*G* fC* A* mC*A*G* fC*mC* | | |
| 21177135.7) | fU*mU* A* fU* G* fC*A* mC*mG*mG | | |
| V117.27 | mC*mA*mU* G*G* fC*mC* fC* mC* A*G* fC* A*G* mC* fU* | 2 | 1A, |
| | mU* fC* A*G* mU* fC* mC* fC* mU*mUmU fC dTdCdl* | | 1B |
| | mU* fC* G*A* mU* G*G* fU* mC* A*G* fC* A* mC* A*G* | | |
| | fC*mC* fU*mU* A* fU* G* mC*A* mC*mG*mG | | |
| V117.28 | mC*mA*mU* G*G* fC*mC* fC* mC* A*G* fC* A*G* mC* fU* | 3 | 1A, |
| | mU* fC* A*G* mU* fC* mC* fC* mU*mU*mU* fC* dTdCdl | | 1B |
| | mU fC G*A* mU* G*G* fU* mC* A*G* fC* A* mC* A*G* | | |
| | fC*mC* fU*mU* A* fU* G* mC*A* mC*mG*mG | | |
| V117.29 | mC*mA*mU* G*G* fC*mC* fC* mC* A*G* fC* A*G* mC* fU* | 4 | 1A, |
| | mU* fC* A*G* mU* fC* mC* fC* mU*mU*mU* fC* dTdC*dl* | | 1B |
| | mU* fC* G*A* mU* G*G* fU* mC* A*G* fC* A* mC* A*G* | | |
| | fC*mC* fU*mU* A* fU* G* mC*A* mC*mG*mG | | |
| V117.30 | mC*mA*mU* G*G* fC*mC* fC* mC* A*G* fC* A*G* mC* fU* | 5 | 1A, |
| | mU* fC* A*G* mU* fC* mC* fC* mU*mUmU fC* dTdC*dl* | | 1B |
| | mU fC G*A* mU* G*G* fU* mC* A*G* fC* A* mC* A*G* | | |
| | fC*mC* fU*mU* A* fU* G* mC*A* mC*mG*mG | | |
| V117.33 | mC*mA*mU* G*G* fC*mC* fC* mC* A*G* fC* A*G* mC* fU* | 6 | 1A, |
| | mU* fC* A*G* mU* fC* mC* fC* mU*mU*mU* fC* dTdC*dl | | 1B |
| | mU fC GA* mU* G*G* fU* mC* A*G* fC* A* mC* A*G* | | |
| | fC*mC* fU*mU* A* fU* G* mC*A* mC*mG*mG | | |
| V117.34 | mC*mA*mU* G*G* fC*mC* fC* mC* A*G* fC* A*G* mC* fU* | 7 | 1A, |
| | mU* fC* A*G* mU* fC* mC* fC* mU*mUmU fC* dTdC*dl | | 1B |
| | mU fC GA* mU* G*G* fU* mC* A*G* fC* A* mC* A*G* | | |
| | fC*mC* fU*mU* A* fU* G* mC*A* mC*mG*mG | | |
| V117.35 | mC*mA*mU* G*G* fC*mC* fC* mC* A*G* fC* A*G* mC* fU* | 8 | 1A, |
| | mU* fC* A*G* mU* fC* mC* fC* mU*mUmU fC* dTdCdl | | 1B |
| | mU fC GA* mU* G*G* fU* mC* A*G* fC* A* mC* A*G* | | |
| | fC*mC* fU*mU* A* fU* G* mC*A* mC*mG*mG | | |
| V117.39 | mC*mA*mU*G*G* fC*mC*fC*mC*A*G* | 9 | 1A, |
| | fC*A*G*mC*fU*mU*fC*A*G*mU*fC*mC*fC*mU*mUmUfC* | | 1B |
| | dT*dC*dl | | |
| | mUfCG*A*mU*G*G*fU*mC*A*G*fC*A*mC*A*G*fC*mC*fU* | | |
| | mU*A*fU*G*mC*A*mC*mG*mG | | |
| V117.40 | mC*mA*mU*G*G*fC*mC* fC*mC*A*G*fC*A*G*mC* | 10 | 1A, |
| | fU*mU*fC*A*G*mU*fC*mC*fC*mU*mUmUfC* dT*dC*dl* | | 1B |
| | mU fC | | |
| | GA*mU*G*G*fU*mC*A*G*fC*A*mC*A*G*fC*mC*fU*mU*A* | | |
| | fU*G*mC*A*mC*mG*mG | | |

mN = 2′-O-methyl,
fN = 2′fluoro,
N = 2′OH (ribo),
dN = 2′H(DNA)
* = phosphorothioate linkage

### Example 2: Transfer of the Optimized PS-Linkage Pattern to Oligonucleotides Targeting Endogenous Human STAT1 Y701

The editing efficacy results of Example 2 are shown in FIG. 3A, while the serum half-lives of the constructs are shown in FIG. 3B. The optimal PS linkage pattern surrounding the CBT, which was found for the SERPINA1 E342K target in the embodiment v117.39 (SEQ ID NO:9), was transferred to an embodiment targeting the endogenous human STAT1 transcript inducing the amino acid change Y701C that removes a functionally important phosphotyrosine of the STAT1 protein by RNA editing. Here, it is shown that the optimized PS-linkage pattern from Example 1 could be successfully transferred to a different target. A list of oligonucleotide constructs is provided in Table 2.

10⁵ HeLa cells (Cat. No.: ATCC CCL-2) were seeded in a 24-well plate. After 24 h, cells were forward transfected by diluting 25 pmol construct/well and 1.5 μl/well Lipofectamine RNAiMAX Reagent (ThermoFisher Scientific) in 50 μl Opti-MEM (ThermoFisher Scientific) each and incubated for 5 min at room temperature. After incubation, both solutions were combined to a total volume of 100 μl/well and incubated for an additional 20 min at room temperature. After incubation, the transfection mix was slowly distributed into one well. 24 h after transfection, cells were harvested for RNA isolation and sequencing.

As shown in FIG. 3B and A, v117.29 (Seq. ID No. 13), which contains the optimized PS-linkage pattern from v117.39 (SEQ ID NO:9), outperforms its corresponding construct lacking PS-linkages around the CBT, v117.28 (SEQ ID NO:12, disclosed in patent EP 21177135.7), both in terms of stability in 100% FBS (6 d versus 30 h) and in terms of editing yield (50.5% vs. 40.5%), respectively. We also included the comparison with v117.19 (SEQ ID NO:11), a construct that is only minimally modified, and which shows elevated editing yields compared to v117.28, the more densely modified construct from our prior art (EP 21177135.7). With the optimized PS-pattern, v117.29 reaches editing yields comparable to the much less modified v117.19. Additionally, v117.29 has a 5-fold longer half-life in 100% FBS (6 days) compared to v117.28 (30 h). Clearly, the optimized PS-linkage arrangement showed the same improvement on editing yields and serum half-life in 100% FBS as already seen for the SERPINA1 target (Example 1). Thus, it can be concluded that the optimized PS linkage pattern is transferable to other relevant targets.

TABLE 2
Construct sequences and modifications
used in Example 2.

| | | SEQ | |
|---|---|---|---|
| Construct | Construct Sequence | ID | FIG. |
| Name | (5′ to 3′ direction) | NO: | No. |
| V117.19 | mC*mA*mG*A*C*A*C*A*G*A* | 11 | 2A, |
| | A*A*U*C*A*A*C*U*C*A*G*U* | | 2B |
| | C*U*U*GAU ACA | | |
| | UCC*A*G*U*U*C*C*U*U*U*A* | | |
| | G*G*G*C*C*A*U*C*A*A*G*U | | |
| | *mU*mC*mC | | |
| V117.28 | mC*mA*mG*A* fC*A* fC*A* | 12 | 2A, |
| (disclosed | G*A*A*A* fU*fC* A*A* | | 2B |
| in EP | fC*fU*fC*A*G* fU*fC*fU* | | |
| 21177135.7) | fU*GA fU dAdCdA fU fC | | |
| | fC*A*G* | | |
| | fU*fU*fC*fC*fU*fU*fU* | | |
| | A*G*G*G* fC*fC*A* fU* | | |
| | fC* A*A*G* | | |
| | fU* mU*mC*mC | | |
| V117.29 | mC*mA*mG*A*fC*A*mC*A*G* | 13 | 2A, |
| | A*A*A*fU*mC*A*A*fC*mU* | | 2B |
| | fC*A*G*mU*fC*mU*fU* | | |
| | GAmU*dA*dC*dA mUfC | | |
| | fC*A*G*mU*fU*mC*fC*mU*fU | | |
| | *mU*A*G*G*G*fC*mC*A*fU* | | |
| | mC*A*A*G*fU*mU*mC*mC- | | |
| | GalNAc | | |

mN = 2′-O-methyl,
fN = 2′fluoro,
N = 2′OH (ribo),
dN = 2′H(DNA)
* = phosphorothioate linkage

### Example 3: Transfer of the Optimized PS-Linkage Pattern to of Oligonucleotides Targeting the Disease-Causing W104X Mutation in Marine MECP2

The results of Example 3 are shown in FIG. 4. Editing efficacies of the constructs are shown in FIG. 4A, while the corresponding serum half-lives in 100% FBS are shown in FIG. 4B. The transfer of the PS pattern of v117.39 (SEQ ID NO:9) from the SERPINA1 E342K target to endogenous human STAT1 Y701 proved to be successful. Thus, to further test the transferability of the optimized PS design, it was tested on constructs targeting the W104X mutation in murine MECP2, which is an underlying cause of the severe Rett syndrome. It is shown that the optimized PS-linkage pattern from Example 1 and 2 could be successfully transferred to another target in a clinically relevant sequence context. A list of oligonucleotide constructs used, showing the full modification pattern is provided in Table 3.

5×10⁴ HeLa cells (Cat. No.: ATCC CCL-2) were seeded in a 24-well plate. After 24 h, cells were forward transfected with a plasmid containing the murine MECP2 W104X mutated cDNA. 300 ng plasmid and 0.9 μl FuGENE® 6 (Promega) were each diluted in 50 μl Opti-MEM and incubated for 5 min, then combined and incubated for an additional 20 min. The medium was changed, and the transfection mix evenly distributed into one well. 24 h after plasmid transfection, cells were forward transfected with 25 pmol construct/well and 1.5 μl/well Lipofectamine RNAiMAX Reagent (ThermoFisher Scientific). After 24 h, medium was changed. 48 h after transfection, cells were harvested for RNA isolation and sequencing.

Shown in FIGS. 4A and 4B is the comparison of editing yields and half-lives in 100% FBS of the three different constructs tested, respectively. V120.17 (SEQ ID NO:14) is a construct that has PS linkages only at positions a and j, and performs well in editing (ca. 50%), but is unstable in 100% FBS (half-life of ca. 48 h). On the other hand, the construct v120.24 (SEQ ID NO:16), which has PS linkages at every position, has greatly enhanced serum half-life (ca. 7 days), but also lost significant editing yield, down to almost half of what was observed for v120.17 (ca. 30%). However, when applying the optimized PS-linkages pattern from v117.39 (SEQ ID NO:9), the construct v120.23 (Seq. ID No. 15) reaches the same editing efficacy as v120.17 (ca. 50%), while simultaneously achieving a 1.5-fold improvement of the half-life in 100% FBS (ca. 72 h). This underlines the power of precise positioning of the PS-linkages, even for constructs of different design and length (compare constructs from Example 1 and 2 with Example 3), further emphasizing the transferability of the pattern to other targets and oligonucleotide sequence designs.

TABLE 3
Construct sequences and modifications
used in Example 3.

| | | SEQ | |
|---|---|---|---|
| Construct | Construct Sequence | ID | FIG. |
| Name | (5′ to 3′ direction) | NO: | No. |
| V120.21 | mU*mC*mG*G* fC*fC* A* | 14 | 3A, |
| (disclosed | G*A*fC*fU*fU*fC*fC*fU* | | 3B |
| in EP | fU*fU* G* fU*fU*fU*A* | | |
| 21177135.7) | A*G*fC*fU*fU*fU*fC*G* | | |
| | fU G fU dCdC A A fC f | | |
| | C*fU*fU*fC* | | |
| | mA*mG*mG | | |
| V120.23 | mU*mC*mG*G* fC*fC* A* | 15 | 3A, |
| | G*A*fC*fU*fU*fC*fC*fU* | | 3B |
| | fU*fU* G* fU*fU*fU*A* | | |
| | A*G*fC*fU*fU*fU*fC*G* | | |
| | fU G fU* dC*dC* A A fC | | |
| | fC*fU*fU*f | | |
| | C* mA*mG*mG | | |
| V120.24 | mU*mC*mG*G* fC*fC* A* | 16 | 3A, |
| | G*A*fC*fU*fU*fC*fC*fU* | | 3B |
| | fU*fU* G* fU*fU*fU*A* | | |
| | A*G*fC*fU*fU*fU*fC*G* | | |
| | fU* G* fU* dC*dC* A* | | |
| | A* fC*fC*fU | | |
| | *fU*fC* mA*mG*mG | | |

mN = 2′-O-methyl,
fN = 2′fluoro,
N = 2′OH (ribo),
dN = 2′H(DNA)
* = phosphorothioate linkage

### Example 4: Transfer of the Optimized PS-Linkage Pattern to Oligonucleotides Targeting the Endogenous Human L157 GAPDH Site

The results of Example 4 are shown in FIG. 5. Editing efficacies of the constructs are provided in FIG. 5A, while the corresponding serum half-lives in 100% FBS are shown in FIG. 5B). The optimized PS design was transferred to a construct targeting the endogenous GAPDH transcript at the L157 site. The optimized PS pattern improved half-life stability in 100% FBS about twofold. A list of constructs showing the PS positions used is provided in Table 4.

10⁵ HeLa cells (Cat. No.: ATCC CCL-2) were seeded in a 24-well plate. After 24 h, cells were forward transfected by diluting 25 pmol construct/well and 1.5 μl/well Lipofectamine RNAiMAX Reagent (ThermoFisher Scientific) in 50 μl Opti-MEM (ThermoFisher Scientific) each and incubated for 5 min at room temperature. After incubation, both solutions were combined to a total volume of 100 μl/well and incubated for an additional 20 min at room temperature. After incubation, the transfection mix was slowly distributed into one well. 24 h after transfection, cells were harvested for RNA isolation and sequencing.

FIGS. 5A and 5B show the editing yields and half-life of the constructs in 100% FBS, respectively. Compared to the construct v120.21 (Seq. ID No. 17) without any optimization of the PS linkages in the CBT area, the construct with the optimized PS linkage pattern v120.22 (SEQ ID NO:18) shows a twofold increase of the half-life in 100% FBS. While editing yields do drop compared to v120.21, the editing yields of v120.22 are still twofold higher than v120.23 (SEQ ID NO:19), a construct where all linkages are PS. Given the already long half-life of v120.21 in 100% FBS (72 h) and the low initial editing yield (ca. 25%), the effect of the PS-optimization is less pronounced as for the targets shown in Examples 1, 2 and 3. Nonetheless, it still provides a strong enough effect to underline the transferability and flexibility of this invention.

TABLE 4
Construct sequences and modifications
used in Example 4.

| | | SEQ | |
|---|---|---|---|
| Construct | Construct Sequence | ID | FIG. |
| Name | (5′ to 3′ direction) | NO: | No. |
| V120.21 | mC*mA*mA*A*G*fU*mU*G* | 17 | 4A, |
| (disclosed | fU*mC*A*fU*G*G*A*mU* | | 4B |
| in | G*A*fC*mC*fU*mU*G*G* | | |
| EP | fC*mC*A*G*G*G*GfUG | | |
| 21177135.7) | dCdCdA AGmC*A*G*fU* | | |
| | mU*mG*mG | | |
| V120.22 | mC*mA*mA*A*G*fU*mU*G* | 18 | 4A, |
| | fU*mC*A*fU*G*G*A*mU*G | | 4B |
| | *A*fC*mC*fU*mU*G*G* | | |
| | fC*mC*A*G*G*G*GfUG* | | |
| | dC*dC*dA AGmC*A*G*fU* | | |
| | mU*mG*mG | | |
| V120.23 | mC*mA*mA*A*G*fU*mU*G* | 19 | 4A, |
| | fU*mC*A*fU*G*G*A*mU*G | | 4B |
| | *A*fC*mC*fU*mU*G*G* | | |
| | fC*mC*A*G*G*G*G*fU*G* | | |
| | dC*dC*dA* A*G*mC*A*G* | | |
| | fU*mU*mG*mG | | |

mN = 2′-O-methyl,
fN = 2′fluoro,
N = 2′OH (ribo),
dN = 2′H(DNA)
* = phosphorothioate linkage

### Example 5: Transfer of the Optimized PS-Linkage Design to ASOs Targeting the Disease-Causing G2019S Mutation in Human LRRK2

The results of Example 5 are shown in FIG. 6. The optimized PS design from Example 1 (v117.34 and 117.39 in SERPINA1) was transferred to an oligonucleotide targeting the transiently over expressed LRRK2 transcript bearing the Parkinson's disease causing G2019S mutation. A list of constructs showing the PS positions used is provided in Table 5.

5×10⁴ HeLa cells (Cat. No.: ATCC CCL-2) were seeded in a 24-well plate. After 24 h, cells were forward transfected with a plasmid containing the human LRRK2 G2019S mutated cDNA. 300 ng plasmid and 0.9 μl FuGENE® 6 (Promega) were each diluted in 50 μl Opti-MEM and incubated for 5 min, then combined and incubated for an additional 20 min. The medium was changed, and the transfection mix evenly distributed into one well. 24 h after plasmid transfection, cells were forward transfected with 25 pmol construct/well and 1.5 μl/well Lipofectamine RNAiMAX Reagent (ThermoFisher Scientific). 24 h post-transfection, cells were harvested for RNA isolation and sequencing.

FIG. 6A shows the editing yields. Both PS patterns v117.34 (Seq. ID No. 21) and 117.39 (SEQ ID NO:22) gave comparable or even better editing yields than the earlier oligonucleotide V117.19 (Seq. ID No. 20) lacking PS in the central region.

TABLE 5
Construct sequences and modifications
used in Example 5.

| | | SEQ | |
|---|---|---|---|
| Construct | Construct Sequence | ID | FIG. |
| Name | (5′ to 3′ direction) | NO: | No. |
| V117.19 | mC*mC*mC*C*A*U*U*C*U* | 20 | |
| (disclosed | A*C*A*G*C*A*G*U*A*C*U | | |
| in | *G*A*G*C*A*AUG CCdl | | |
| EP | UAG*U*C*A*G*C*A*A*U*C* | | |
| 21177135.7) | U*U*U*G*C*A*A*U*G*A*U | | |
| | *G*G*mC*mA*mG | | |
| V117.20 | mC*mC*mC*fC* A*mU*fU* | 21 | |
| (disclosed | mC*fU* A*mC* A* G*fC* | | |
| in | A* | | |
| EP | G*mU* A*fC*mU* G* A* | | |
| 21177135.7) | G*fC* A* AmU G dCdCdl | | |
| | fUAG*mU*fC*A*G*mC*A*A* | | |
| | fU*mC*fU*mU*fU*G*mC*A | | |
| | *A*fU*G*A*mU*G*G*mC | | |
| | *mA*mG | | |
| V117.34 | mC*mC*mC*fC*A*mU*fU*mC* | 22 | |
| | fU*A*mC*A*G*fC*A*G*mU | | |
| | *A*fC*mU*G*A*G*fC*A*Am | | |
| | UG* dC*dC*dl fU | | |
| | AGmU*fC*A*G*mC*A*A*fU* | | |
| | mC*mU*mU*mU*G*mC*A*A | | |
| | *fU*G*A*mU*G*G*mC*mA*mG | | |
| V117.39 | mC*mC*mC*fC*A*mU*fU*mC* | 23 | |
| | fU*A*mC*A*G*fC*A*G*mU | | |
| | *A*fC*mU*G*A*G*fC*A*Am | | |
| | UG* dC*dC*dl fU AG* | | |
| | mU*fC*A*G*mC*A*A*fU*mC* | | |
| | mU*mU*mU*G*mC*A*A*fU* | | |
| | G*A*mU*G*G*mC*mA*mG | | |

mN = 2′-O-methyl,
fN = 2′fluoro,
N = 2′OH (ribo),
dN = 2′H(DNA)
* = phosphorothioate linkage

### Example 6: Transfer of the Optimized PS-Linkage Design to ASOs Targeting the Disease-Causing C948Y Mutation in Human CRB1

The results of Example 6 are shown in FIG. 7. The optimized PS design from Example 1 (V117.39 for the SERPINA1 target) was transferred to an oligonucleotide targeting the C948Y mutation site in human CRB1. Mutations in the CRB1 gene are associated with various early-onset retinal dystrophies including Retinitis pigmentosa and Leber congenital amaurosis. Furthermore, oligonucleotides targeting the retina would greatly profit from increased stability, requiring fewer administrations and thus fewer potentially invasive injections into the patients' eye. A list of corresponding constructs used is shown in Table 6.

5×10⁴ HeLa cells (Cat. No.: ATCC CCL-2) were seeded in a 24-well plate. After 24 h, cells were forward transfected with a plasmid containing the human CRB1 C948Y mutated cDNA. 300 ng plasmid and 0.9 μl FuGENE® 6 (Promega) were each diluted in 50 μl Opti-MEM and incubated for 5 min, then combined and incubated for an additional 20 min. The medium was changed, and the transfection mix evenly distributed into one well. 24 h after plasmid transfection, cells were forward transfected with 25 pmol construct/well and 1.5 μl/well Lipofectamine RNAiMAX Reagent (ThermoFisher Scientific). After 24 h, cells were harvested for RNA isolation and sequencing.

FIG. 7A shows the editing yields for the different constructs. For the long, symmetric embodiment with a PS-optimization in the core region, V117.20 (SEQ ID NO:24), only a small drop in editing yields is observed as compared to the unstable construct V117.19 (SEQ ID NO:23) without PS-optimization. For the shorter, asymmetric embodiment V120.17 (SEQ ID NO:25), which also has a PS-optimized core region, editing yields even remain comparable to version V117.19. However, the 100% FBS half-lives of V117.20 (>7 days) and V120.17 (10 h)—shown in FIG. 7B—are greatly increased compared to V117.19 (<1 min). This underlines the influence of stabilizing measures, such as the optimal placement of PS linkages in the core region of the constructs, independent of the construct design.

TABLE 6
Construct sequences and modifications
used in Example 6.

| | | SEQ | |
|---|---|---|---|
| Construct | Construct Sequence | ID | FIG. |
| Name | (5′ to 3′ direction) | NO: | No. |
| V117.19 | mU*mU*mU*G*U*C*C*A*U*U* | 24 | 7 |
| | A*A′A*A*A*C*A*G*C*A*U | | |
| | *U*U*G*C* AAU ACA UUC | | |
| | *A*A*A*U*C*C*U*U*G*A* | | |
| | A*G*C*A*C*C*G*G*C*U*G* | | |
| | G*mC*mA*mC | | |
| V117.20 | mU*mU*mU*G*fU*mC*fC*A* | 25 | 7 |
| | mU*fU*A*A*A*A*A*mC*A*G | | |
| | *fC*A*mU*fU*mU*G*fC* A | | |
| | AmU* dA*dC*dA mUfUfC | | |
| | *A*A*A*mU*fC*mC*fU*mU* | | |
| | G*A*A*G*fC*A*mC*fC*G*G* | | |
| | mC*fU*G*G*mC*mA*mC | | |
| V120.17 | mA*mC*mC*G*fC*mU*fU*mU* | 26 | 7 |
| | G*fU*mC*fC*A*mU*fU*A* | | |
| | A*A*A*A*mC*A*G*fC*A*mU* | | |
| | fU*mU*G*fC* AAmU* | | |
| | dA*dC*dA mUfUfC*A*A*A* | | |
| | mU*mC*mC | | |

mN = 2′-O-methyl,
fN = 2′fluoro,
N = 2′OH (ribo),
dN = 2′H(DNA)
* = phosphorothioate linkage

### Example 7: Transfer of the Optimized PS-Linkage Design to ASOs Targeting the Endogenous T41 Site on Murine CTNNB1

The results of Example 7 are shown in FIG. 8. The optimized PS design from Example 1 (V117.39 for the SERPINA1 target) was transferred to an oligonucleotide targeting the T41 site in murine CTNNB1. The encoded protein, β-catenin, is a key component in cell growth and tissue homeostasis and is degraded upon phosphorylation at the T41 site. A mutation at the T41 site can thus prolong β-catenin's presence in the cell and effectively accelerate i.e. tissue regeneration. A list of corresponding constructs is presented in Table 7.

10⁵ mouse embryonic fibroblast (MEF) cells were seeded in a 24-well plate. After 24 h, cells were forward transfected by diluting 25 pmol construct/well and 1.5 μl/well Lipofectamine RNAiMAX Reagent (ThermoFisher Scientific) in 50 μl Opti-MEM (ThermoFisher Scientific) each and incubated for 5 min at room temperature. After incubation, both solutions were combined to a total volume of 100 μl/well and incubated for an additional 20 min at room temperature. After incubation, the transfection mix was slowly distributed into one well. 24 h after transfection, cells were harvested for RNA isolation and sequencing.

FIG. 8A shows the editing yields of v117.20 (SEQ ID NO:26), which reaches about 20%, while FIG. 8B shows the half-life of the construct in 100% FBS (>7 days). The murine CTNNB1 T41 site thus provides another example where the optimized PS linkage placement can be applied.

TABLE 7
Construct sequences and modifications used
in Example 7.

| | | SEQ | |
|---|---|---|---|
| Construct | Construct Sequence | ID | FIG. |
| Name | (5′ to 3′ direction) | NO: | No. |
| V117.20 | mG*mC*mC*fC*mU*fU*G*mC* | 27 | 8 |
| | fC*A*mC*fU*mC*A*G*G* | | |
| | G*A*A*G*G*A*G*fC*mU* | | |
| | GfUG* dG*dC*dl GmUG* | | |
| | G*fC*A*mC*fC*A*G*A*A* | | |
| | mU*G*G*A*fU*mU*fC*mC* | | |
| | A*G*A*A*fU*mC*mC*mA | | |

mN = 2′-O-methyl,
fN = 2′fluoro,
N = 2′OH (ribo),
dN = 2′H(DNA)
* = phosphorothioate linkage

The sequences disclosed herein are also shown in the enclosed sequence listing. The sequence listing shows, however, only the sequence of nucleotides whereas the modification of the nucleotides and of the bonds between the nucleotides is not shown in the sequence listing. The relevant sequences are disclosed in the tables above.

1. A chemically modified oligonucleotide for use in site-directed A-to-I editing of a target RNA inside a cell with endogenous ADAR, comprising a sequence with a length of 11 to 100 nucleotides capable of binding to a target sequence in the target RNA, with a Central Base Triplet of 3 nucleotides with the central nucleotide opposite to the target adenosine in the target RNA, which is to be edited to an inosine, whereby the core sequence has the following Formula 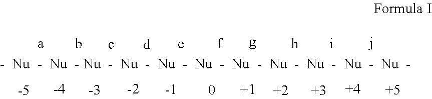
wherein Nu stands for a nucleotide having a sugar moiety which may be modified, the numbers below the nucleotide sequence designate the position of the nucleotides adjacent to the central nucleotide of the Central Base Triplet having the number 0 whereby the negative numbers designate the 5′ end and the positive number designate the 3′ end of the oligonucleotide and wherein a-j designate the nature of the linkage between the single nucleotides whereby at least linkages a, d, and e are phosphorothioate linkages and whereby at least 2 linkages are a phosphate linkage(s).

2. A chemically modified oligonucleotide according to claim 1 characterized in that the linkages h and i are phosphate linkages.

3. A chemically modified oligonucleotide according to claim 1 characterized in that also the linkage f is a phosphorothioate linkage.

4. A chemically modified oligonucleotide according to claim 1 characterized in that also the linkage j is a phosphorothioate linkage.

5. A chemically modified oligonucleotide according to claim 1 characterized in that also the linkage g is a phosphorothioate linkage.

6. A chemically modified oligonucleotide according to claim 1 characterized in that also the linkages f and j are phosphorothioate linkages.

7. A chemically modified oligonucleotide according to claim 1 characterized in that also the linkages b and/or c are phosphorothioate linkages.

8. A chemically modified oligonucleotide according to claim 1 characterized in that at least one linkage is a stereopure phosphorothioate linkage.

9. A chemically modified oligoribonucleotide according to claim 1 characterized in that
a) at least 90% of the pyrimidine nucleosides outside the Central Base Triplet are chemically modified, either at the 2′ position of the sugar moiety, or are deoxyribonucleosides, or a combination thereof,
b) no more than 6 consecutive nucleosides are chemically modified with 2″-O-methyl at the 2″ position of the sugar moiety,
c) at least two of the three nucleosides of the Central Base Triplet are chemically modified at the 2′ position of the sugar moiety, or are deoxyribonucleosides, or a combination thereof.

10. A chemically modified oligonucleotide according to claim 1 characterized in that at least 50%, more preferably at least 80% of the nucleotides from Nu −5 to +5 are modified independently from another by one of the following modifications at the 2″ position of the ribose: 2″-Fluoro, or 2″-O-methyl, or 2″-H (desoxy).

11. A chemically modified oligonucleotide according to claim 1 characterized in that all three nucleotides Nu −1, 0, and +1 have an H residue at the 2″ position of the ribose.

12. A chemically modified oligonucleotide according to claim 1 characterized in that the nucleotide Nu +1 carries an N-heterocyclic base, preferably a purine derivative, more preferably a hypoxanthine base, or a derivative thereof.

13. A chemically modified oligonucleotide according to claim 1 characterized in that the nucleotide Nu 0 carries a nucleobase based on an N-heterocycle, preferably a pyrimidine or pyridine, more preferably a cytosine, or a derivative thereof.

14. A chemically modified oligonucleotide according to claim 1 characterized that it has a length of 20 to 80 nucleotides.

15. A chemically modified oligonucleotide according to claim 1 characterized that it has a length of 30 to 60 nucleotides.

16. A chemically modified oligonucleotide according to claim 1, wherein, within the core sequence of formula I, only a, d, e and optionally up to three linkages selected from b, c, f, g and j, are phosphorothioate linkages.

17. A chemically modified oligonucleotide according to claim 16, wherein within the core sequence of formula I, only a, d, e and f are phosphorothioate linkages.


SEQUENCE LISTING (27 sequences)

SEQ ID NO: 1 - DNA, 59 bp, synthetic construct
  source 1..59: mol_type = other DNA
  misc_feature 1..59: note = V117.26
  modified_base 31: mod_base = i
  misc_feature order(1..28,32..59): note = RNA
  misc_feature 29..31: note = DNA
catggccccagcagcttcagtccctttctcntcgatggtcagcacagccttatgcacgg

SEQ ID NO: 2 - DNA, 59 bp, synthetic construct; identical to SEQ ID NO: 1
  source 1..59: mol_type = other DNA
  misc_feature 1..59: note = V117.27
  modified_base 31: mod_base = i
  misc_feature order(1..28,32..59): note = RNA
  misc_feature 29..31: note = DNA

SEQ ID NO: 3 - DNA, 59 bp, synthetic construct; identical to SEQ ID NO: 1
  source 1..59: mol_type = other DNA
  misc_feature 1..59: note = V117.28
  modified_base 31: mod_base = i
  misc_feature order(1..28,32..59): note = RNA
  misc_feature 29..31: note = DNA

SEQ ID NO: 4 - DNA, 59 bp, synthetic construct; identical to SEQ ID NO: 1
  misc_feature 1..59: note = V117.29
  source 1..59: mol_type = other DNA
  modified_base 31: mod_base = i
  misc_feature order(1..28,32..59): note = RNA
  misc_feature 29..31: note = DNA

SEQ ID NO: 5 - DNA, 59 bp, synthetic construct; identical to SEQ ID NO: 1
  source 1..59: mol_type = other DNA
  misc_feature 1..59: note = V117.30
  modified_base 31: mod_base = i
  misc_feature order(1..28,32..59): note = RNA
  misc_feature 29..31: note = DNA

SEQ ID NO: 6 - DNA, 59 bp, synthetic construct; identical to SEQ ID NO: 1
  source 1..59: mol_type = other DNA
  misc_feature 1..59: note = V117.33
  modified_base 31: mod_base = i
  misc_feature order(1..28,32..59): note = RNA
  misc_feature 29..31: note = DNA

SEQ ID NO: 7 - DNA, 59 bp, synthetic construct; identical to SEQ ID NO: 1
  source 1..59: mol_type = other DNA
  misc_feature 1..59: note = V117.34
  modified_base 31: mod_base = i
  misc_feature order(1..28,32..59): note = RNA
  misc_feature 29..31: note = DNA

SEQ ID NO: 8 - DNA, 59 bp, synthetic construct; identical to SEQ ID NO: 1
  misc_feature 1..59: note = V117.35
  source 1..59: mol_type = other DNA
  modified_base 31: mod_base = i
  misc_feature order(1..28,32..59): note = RNA
  misc_feature 29..31: note = DNA

SEQ ID NO: 9 - DNA, 59 bp, synthetic construct; identical to SEQ ID NO: 1
  misc_feature 1..59: note = V117.39
  source 1..59: mol_type = other DNA
  modified_base 31: mod_base = i
  misc_feature order(1..28,32..59): note = RNA
  misc_feature 29..31: note = DNA

SEQ ID NO: 10 - DNA, 59 bp, synthetic construct; identical to SEQ ID NO: 1
  misc_feature 1..59: note = V117.40
  source 1..59: mol_type = other DNA
  modified_base 31: mod_base = i
  misc_feature order(1..28,32..59): note = RNA
  misc_feature 29..31: note = DNA

SEQ ID NO: 11 - RNA, 59 nt, synthetic construct
  source 1..59: mol_type = other RNA
  misc_feature 1..59: note = V117.19
cagacacagaaatcaactcagtcttgatacatccagttcctttagggccatcaagttcc

SEQ ID NO: 12 - DNA, 59 bp, synthetic construct
  misc_feature 1..59: note = V117.28
  source 1..59: mol_type = other DNA
  misc_feature order(1..28,32..59): note = RNA
  misc_feature 29..31: note = DNA
cagacacagaaatcaactcagtcttgatacatccagttcctttagggccatcaagttcc

SEQ ID NO: 13 - DNA, 59 bp, synthetic construct; identical to SEQ ID NO: 12
  misc_feature 1..59: note = V117.29
  source 1..59: mol_type = other DNA
  misc_feature order(1..28,32..59): note = RNA
  misc_feature 29..31: note = DNA

SEQ ID NO: 14 - DNA, 45 bp, synthetic construct
  misc_feature 1..45: note = V120.21
  source 1..45: mol_type = other DNA
  misc_feature order(1..33,36..45): note = RNA
  misc_feature 34..35: note = DNA
tcggccagacttcctttgtttaagctttcgtgtccaaccttcagg

SEQ ID NO: 15 - DNA, 45 bp, synthetic construct; identical to SEQ ID NO: 14
  misc_feature 1..45: note = V120.23
  source 1..45: mol_type = other DNA
  misc_feature order(1..33,36..45): note = RNA
  misc_feature 34..35: note = DNA

SEQ ID NO: 16 - DNA, 45 bp, synthetic construct; identical to SEQ ID NO: 14
  misc_feature 1..45: note = V120.24
  source 1..45: mol_type = other DNA
  misc_feature order(1..33,36..45): note = RNA
  misc_feature 34..35: note = DNA

SEQ ID NO: 17 - DNA, 45 bp, synthetic construct
  misc_feature 1..45: note = V120.21
  source 1..45: mol_type = other DNA
  misc_feature order(1..33,37..45): note = RNA
  misc_feature 34..36: note = DNA
caaagttgtcatggatgaccttggccaggggtgccaagcagttgg

SEQ ID NO: 18 - DNA, 45 bp, synthetic construct; identical to SEQ ID NO: 17
  misc_feature 1..45: note = V120.22
  source 1..45: mol_type = other DNA
  misc_feature order(1..33,37..45): note = RNA
  misc_feature 34..36: note = DNA

SEQ ID NO: 19 - DNA, 45 bp, synthetic construct; identical to SEQ ID NO: 17
  misc_feature 1..45: note = V120.23
  source 1..45: mol_type = other DNA
  misc_feature order(1..33,37..45): note = RNA
  misc_feature 34..36: note = DNA

SEQ ID NO: 20 - DNA, 59 bp, synthetic construct
  misc_feature 1..59: note = V117.19
  source 1..59: mol_type = other DNA
  modified_base 31: mod_base = i
  misc_feature order(1..30,32..59): note = RNA
  misc_feature 31: note = DNA
ccccattctacagcagtactgagcaatgccntagtcagcaatctttgcaatgatggcag

SEQ ID NO: 21 - DNA, 59 bp, synthetic construct; identical to SEQ ID NO: 20
  misc_feature 1..59: note = V117.20
  source 1..59: mol_type = other DNA
  modified_base 31: mod_base = i
  misc_feature order(1..28,32..59): note = RNA
  misc_feature 29..31: note = DNA

SEQ ID NO: 22 - DNA, 59 bp, synthetic construct; identical to SEQ ID NO: 20
  misc_feature 1..59: note = V117.34
  source 1..59: mol_type = other DNA
  modified_base 31: mod_base = i
  misc_feature order(1..28,32..59): note = RNA
  misc_feature 29..31: note = DNA

SEQ ID NO: 23 - DNA, 59 bp, synthetic construct; identical to SEQ ID NO: 20
  misc_feature 1..59: note = V117.39
  source 1..59: mol_type = other DNA
  modified_base 31: mod_base = i
  misc_feature order(1..28,32..59): note = RNA
  misc_feature 29..31: note = DNA

SEQ ID NO: 24 - RNA, 59 nt, synthetic construct
  source 1..59: mol_type = other RNA
  misc_feature 1..59: note = V117.19
tttgtccattaaaaacagcatttgcaatacattcaaatccttgaagcaccggctggcac

SEQ ID NO: 25 - DNA, 59 bp, synthetic construct
  misc_feature 1..59: note = V117.20
  source 1..59: mol_type = other DNA
  misc_feature order(1..28,32..59): note = RNA
  misc_feature 29..31: note = DNA
tttgtccattaaaaacagcatttgcaatacattcaaatccttgaagcaccggctggcac

SEQ ID NO: 26 - DNA, 45 bp, synthetic construct
  misc_feature 1..45: note = V120.17
  source 1..45: mol_type = other DNA
  misc_feature order(1..33,37..45): note = RNA
  misc_feature 34..36: note = DNA
accgctttgtccattaaaaacagcatttgcaatacattcaaatcc

SEQ ID NO: 27 - DNA, 59 bp, synthetic construct
  misc_feature 1..59: note = V117.20
  source 1..59: mol_type = other DNA
  modified_base 31: mod_base = i
  misc_feature order(1..28,32..59): note = RNA
  misc_feature 29..31: note = DNA
gcccttgccactcagggaaggagctgtggcngtggcaccagaatggattccagaatcca